(19) United States
(12) Patent Application Publication
(10) Pub. No.: US 2026/0217761 A1
(43) Pub. Date: Jul. 30, 2026

(54) ANTIMICROBIAL PEPTIDE RICH IN TRYPTOPHAN AND HAVING SELF-ASSEMBLY PROPERTIES, AND USE THEREOF

(71) Applicant: NANOQURE BIOTECH (SHENZHEN) CO., LTD., Guangdong (CN)

(72) Inventors: Wenjing ZHAO, Guangdong (CN); Shuli CHOU, Guangdong (CN); Xiangyu MOU, Guangdong (CN); Huating GUO, Guangdong (CN); Shiqing ZHANG, Guangdong (CN)

(21) Appl. No.: 19/142,123

(22) PCT Filed: Dec. 20, 2023
(86) PCT No.: PCT/CN2023/140062

§ 371 Date: Jun. 20, 2025

(30) Foreign Application Priority Data
Dec. 21, 2022 (CN) 202211648080.2

(51) Int. Cl. C07K 7/06; A61K 38/00; A61P 31/04

(52) U.S. Cl. CPC C07K 7/06; A61P 31/04; A61K 38/00

(57) ABSTRACT

Provided is a short-chain antimicrobial peptide rich in tryptophan and having self-assembly properties. The short-chain antimicrobial peptide is composed of tryptophan (W) and positively charged amino acids, has self-assembly properties, and can be used as a broad-spectrum antimicrobial peptide against microbial infections, such as co-infections against Pseudomonas aeruginosa and Staphylococcus aureus.

### TECHNICAL FIELD

The present invention belongs to the field of biotechnology, and relates to a new antimicrobial peptide and its use, especially an antimicrobial peptide against bacterial infections.

### BACKGROUND ART

Multidrug resistant (MDR) bacterial infections are a serious global health problem. A recent Global Burden of Disease study estimated that in 2019, the number of deaths associated with antibiotic-resistant bacterial infections was 4.95 million, and the number of deaths directly caused by antibiotic-resistant bacterial infections was 1.27 million [1]. Multidrug resistant bacterial infections have become one of the leading causes of global death. Multidrug resistant bacteria often exist in the form of biofilms. A biofilm is a bacterial aggregate with a complex three-dimensional structure, wrapped by water and extracellular polymers. Biofilms exacerbate the development of antibiotic resistance by altering the microenvironment where cells replicate, slowing down the penetration of antibiotics into cells, and inducing highly resistant phenotypes, etc. [2]. Compared with biofilms composed of a single species of bacterium, mixed biofilms of multiple bacteria can lead to the transfer of drug resistance genes between bacterial species, which further promotes the resistance of biofilm to antibiotics. For example, Staphylococcus aureus increases the resistance of Pseudomonas aeruginosa to tobramycin [3], and in turn, Pseudomonas aeruginosa enhances the resistance of Staphylococcus aureus to vancomycin [4]. Pseudomonas aeruginosa and Staphylococcus aureus often coexist in patients with chronic infections and cystic fibrosis. Such co-infections lead to more severe conditions and higher medical costs [5-7]. Therefore, an ideal antimicrobial preparation should be effective not only against suspended bacteria but also against bacteria in mixed-species biofilms.

Antibacterial peptides, antimicrobial polypeptides, or antimicrobial peptides (AMPs) are one of the most promising alternatives to traditional antibiotics and have shown great potential in treating multidrug resistant bacterial infections. However, the high production cost is one of the important factors limiting their clinical application development. Antimicrobial peptides generally contain 10-100 amino acids. Although the synthesis cost of short peptides is relatively low, their antimicrobial activity is also relatively low [8]. In an antimicrobial peptide library containing 3,324 sequences, only two of the 72 sequences that are effective against biofilms are short peptides with less than 10 amino acids [9]. Therefore, improving the antimicrobial activity of short-chain antimicrobial peptides is an urgent problem to be solved in the current development of antimicrobial peptides.

Previous studies have shown that after antimicrobial peptides self-assemble into nanostructures, their antimicrobial activity can be enhanced by prolonging their half-life and increasing the local concentration. Other studies have shown that a structure called “tryptophan zipper” or “π-π stacking bond” can be formed between tryptophan groups and other tryptophan groups, which can promote the occurrence of intermolecular self-assembly. Therefore, in the present invention, we provide a short-chain antimicrobial peptide rich in tryptophan and having self-assembly activities, which provides a potential candidate drug for multidrug resistant bacterial infections.

### OBJECT OF INVENTION

The present invention aims to provide a potential candidate drug for multidrug resistant bacterial infections.

### SUMMARY OF INVENTION

To solve the above technical problem, the present invention provides a short-chain antimicrobial peptide rich in tryptophan and having self-assembly properties.

The present invention shows that the antimicrobial peptide is a broad-spectrum antimicrobial peptide.

According to the present invention, the antimicrobial peptide is a centrally symmetric 9-peptide (which may contain either L-amino acid and/or D-amino acid) and is composed of tryptophan (W) and positively charged amino acids, wherein the number of positively charged amino acids is 3-6, such as 3, 4, 5 and 6, preferably 4 or 6, and more preferably 4.

According to the present invention, the percentage of hydrophobic amino acids in the antimicrobial peptide is 30-60%, preferably 50% or more, or even 55% or more; and/or the hydrophobicity is −0.1-0.8, preferably 0.6 or more; and/or the hydrophobic moment is 0.01-1.8, preferably 0.5 or more, more preferably 1.2, or even 1.7 or more.

According to the present invention, the positively charged amino acids are selected from arginine (R) and lysine (K).

According to the present invention, the antimicrobial peptide contains a single tryptophan (W) motif, a double tryptophan motif (WW), a triple tryptophan motif (WWW), or a combination thereof, which is evenly separated by positively charged amino acids (+).

According to the present invention, the antimicrobial peptide has a primary symmetric structure with the triple tryptophan (WWW) at the center, or a primary symmetric structure with a tryptophan at the center and double tryptophan (WW) at both ends. Preferably, the antimicrobial peptide contains the double tryptophan motif (WW) at both ends.

According to the present invention, antimicrobial peptides include, but are not limited to, the following general formulae:
Single W motif, such as W+++W+++W, +W++W++W+, ++W+W+W++; WW motif or its combination with a single W motif, such as WW+++++WW, WW+W+W+WW, +WW+++WW+, ++WW+WW++, WW++W++WW (for example, Z3 of the present invention: WWRRWRRWW (SEQ ID NO: 3) and Z4: WWKKWKKWW (SEQ ID NO: 4)), +WW+W+WW+,
WWW motif or its combination with a single W motif or WW motif, such as +++WWW+++ (for example, Z2 of the present invention: RRRWWWRRR (SEQ ID NO: 2)), +W+WWW+W+ (for example, Z1 of the present invention: KWKWWWKWK (SEQ ID NO: 1), WWW+W+WWW, WWW+++WWW, WW+WWW+WW, +WWW+WWW+;
wherein “W” represents tryptophan, and “+” represents a positively charged amino acid, such as arginine (R) or lysine (K). Preferably, the “+” in the same general formula represents the same positively charged amino acid.

As a preferred embodiment, the antimicrobial peptide is selected from any one of SEQ ID NOs: 1-4 and a variant thereof containing D-amino acid, preferably SEQ ID NO: 4 (Z4) or Z4-D.

The present invention also provides a pharmaceutical composition comprising the above-mentioned antimicrobial peptide.

The present invention also provides the use of the above-mentioned antimicrobial peptide against microbial co-infections, such as against the co-infections of Pseudomonas aeruginosa-Staphylococcus aureus. Alternatively, it provides the use of the above-mentioned antimicrobial peptide in the preparation of a medicament against microbial co-infections.

The present invention also provides the use of the above-mentioned antimicrobial peptide for the treatment of multidrug resistant bacterial infections, or the use of the above-mentioned antimicrobial peptides in the preparation of a medicament for the treatment of multidrug resistant bacterial infections.

### Technical Effect

This application designs a short-chain polypeptide rich in tryptophan and having self-assembly properties, which exhibits broad-spectrum antibacterial properties, especially Z4. The minimum inhibitory concentration (MIC) of polypeptide Z4 against a mixed bacterial solution of Pseudomonas aeruginosa and Staphylococcus aureus is 4 μM. Compared with gentamicin, polypeptide Z4 has a faster bactericidal rate against Pseudomonas aeruginosa (P. aeruginosa), while there is no significant difference in the bactericidal rate against Staphylococcus aureus (S. aureus). In addition, polypeptide Z4 can remove more than 50% of the biofilm when the concentration reaches 8 M, which is much better than the 32 M required for gentamicin. This means that polypeptide Z4 has good and direct anti-biofilm activity.

### FIGURES AND BRIEF DESCRIPTIONS THEREOF

To more clearly describe the technical solutions of the present invention, a brief introduction will be given below in combination with the attached figures. Obviously, these figures only represent some specific embodiments recorded in this application. The present invention includes, but is not limited to, these figures.

FIG. 1 illustrates the minimum inhibitory concentration (MIC) of the antimicrobial peptides of the present invention against various bacteria;

FIG. 2 illustrates the minimum inhibitory concentration (MIC) of the antimicrobial peptides of the present invention against a mixed bacterial solution of Pseudomonas aeruginosa and Staphylococcus aureus;

FIG. 3 illustrates the spectral analysis results of polypeptides Z1-Z4;

FIG. 4 illustrates the SEM image (A) and the distribution of micelle diameters (B) of polypeptide Z4 at MIC;

FIG. 5 illustrates the killing curves of the polypeptide Z4 of the present invention against P. aeruginosa (A) and S. aureus (B) at 1 times MIC, and the killing curves against P. aeruginosa (C) and S. aureus (D) at 2 times MIC, respectively; wherein FIG. 5A illustrates the survival number (CFU/mL) of P. aeruginosa after treatment with 1×MIC of Z4 or gentamicin, FIG. 5B illustrates the survival number (CFU/mL) of S. aureus after treatment with 1×MIC of Z4 or gentamicin, FIG. 5C illustrates the survival number (CFU/mL) of P. aeruginosa after treatment with 2×MIC of Z4 or gentamicin, and FIG. 5D illustrates the survival number (CFU/mL) of S. aureus after treatment with 2×MIC of Z4 or gentamicin;

FIG. 6 illustrates the removal effect (%) of the polypeptide Z4 of the present invention or gentamicin on the formed biofilm;

FIG. 7 illustrates the in vivo antimicrobial activity of the polypeptide Z4 of the present invention, wherein FIG. 7C illustrates the bactericidal activity (CFU) of Z4 or PBS against P. aeruginosa and S. aureus in the catheter biofilm, FIG. 7D illustrates the IL-6 content (pg/g) in the skin tissue at the infected site of mice, and FIG. 7F illustrates the number of inflammatory cells in the sections of skin tissue at the infected site of mice.

FIG. 8 illustrates that the dextrorotatory isomer Z4-D of the polypeptide Z4 has a strong ability to resist protease digestion.

### SPECIFIC MODE FOR CARRYING OUT THE INVENTION

To further understand the present invention, preferred embodiments of the present invention will be described below in combination with Examples. These descriptions merely illustrate the features and advantages of the technical solutions of the present invention, rather than limiting the scope of protection of the present invention.

### Example 1

### Preparation of Polypeptides Z1-Z4:

Polypeptides Z1-Z4 can be ordered from a polypeptide synthesis company or prepared according to the following conventional preparation methods.
1. The synthesis proceeds one by one from the C-terminus to the N-terminus according to the amino acid sequence of the antimicrobial peptide, with a polypeptide synthesizer. First, Fmoc-X (X is the first amino acid at the C-terminus of each antimicrobial peptide) is connected to the AAM/MBHA resin, and then the Fmoc group is removed to obtain the X-AAM/MBHA resin; then Fmoc-Y-Trt-OH (9-fluorenylmethoxycarboxyl-trimethyl-Y, wherein Y is the second amino acid at the C-terminus of each antimicrobial peptide) is connected; following this procedure, synthesis proceeds in sequence from the C-terminus to the N-terminus until the synthesis is completed, and the side-chain protected peptide resin with the Fmoc group removed is obtained;
2. A cleavage reagent is added to the peptide resin obtained above, reacted at 20° C. in dark for 2 h, and then filtered; the precipitate is washed with TFA (trifluoroacetic acid), the washed solution is mixed with the filtrate, concentrated with a rotary evaporator, then added with about 10 times the volume of pre-cooled anhydrous ether, precipitated at −20° C. for 3 h, and a white powder will precipitate. Centrifuge at 2500 g for 10 min, collect the precipitate, wash the precipitate with anhydrous ether again, and dry it under vacuum to obtain the polypeptide. The cleavage reagent is a mixture of TFA, water and TIS (triisopropylsilyl chloride) in a mass ratio of 95:2.5:2.5;
3. The column is equilibrated with 0.2 mol/L sodium sulfate (adjusted to pH 7.5 with phosphoric acid) for 30 min. The polypeptide is dissolved in a 90% acetonitrile aqueous solution and filtered. A C18 reverse-phase normal-pressure column is used, with gradient elution (the eluent is a mixture of methanol and sodium sulfate aqueous solution in a volume ratio of 30:70 to 70:30), at a flow rate of 1 mL/min and a detection wavelength of 220 nm. The main peak fraction is collected and freeze-dried. Then, further purification is performed with a reverse-phase C18 column. Eluent A is a 0.1% TFA/aqueous solution; eluent B is a 0.1% TFA/acetonitrile solution, the elution concentration is 25% B~40% B and 75% A 60% A, the elution time is 12 min, and the flow rate is 1 mL/min. Then, the main peak fraction is collected and freeze-dried as above.
4. Identification of antimicrobial peptides: The physicochemical parameters of antimicrobial peptides are shown in Table 1.

TABLE 1
Design of antimicrobial peptides and their main physicochemical parameters

| Name | | | | | Percentage | | |
|---|---|---|---|---|---|---|---|
| of | | Theoretical | Actual | | of | | Hydro- |
| poly- | | molecular | molecular | Charge | hydrophobic | | phobic |
| peptides | Sequence | weight | weight | number | amino acids | Hydrophobicity | moment |
| Z1 | KWKWWWKWK-NH₂ | 1461.75 | 1460.81 | +4 | 55.50% | 0.61 | 0.51 |
| | (SEQ ID NO: 1) | | | | | | |
| Z2 | RRRWWWRRR-NH₂ | 1513.76 | 1512.81 | +6 | 33.30% | 0.78 | 0.01 |
| | (SEQ ID NO: 2) | | | | | | |
| Z3 | WWRRWRRWW-NH₂ | 1573.8 | 1572.86 | +4 | 55.50% | −0.13 | 1.29 |
| | (SEQ ID NO: 3) | | | | | | |
| Z4 | WWKKWKKWW-NH₂ | 1461.75 | 1460.81 | +4 | 55.50% | 0.61 | 1.71 |
| | (SEQ ID NO: 4) | | | | | | |

### Example 2

### Determination of the Antimicrobial Activity of Polypeptides Z1-Z4:

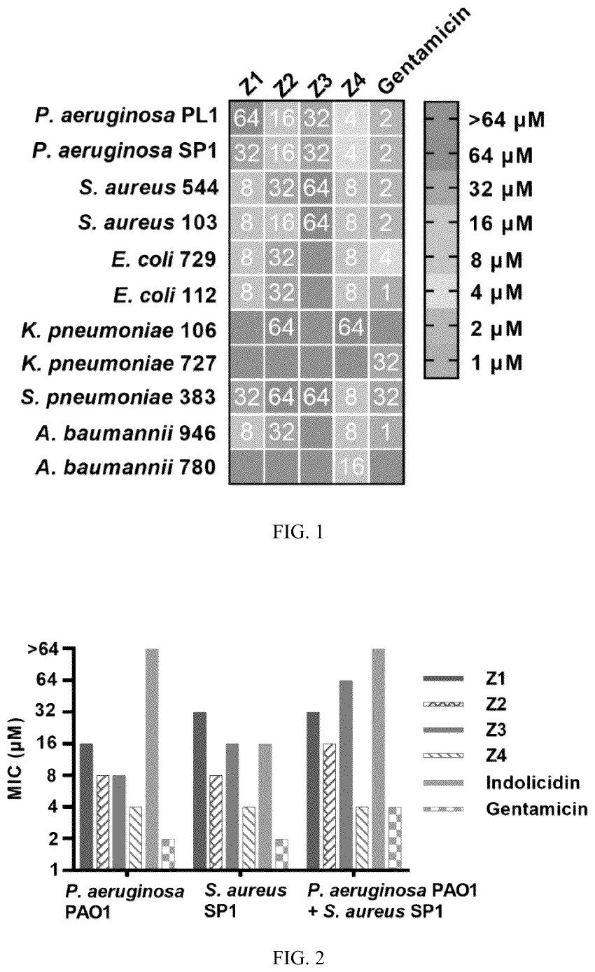

A. The antimicrobial peptide was prepared into a certain stock solution for use. 0.01% acetic acid (containing 0.2% BSA) was used as the diluent, and a series of gradient antimicrobial peptide solutions were prepared sequentially by a two-fold dilution method.
B. 100 μL of the above-mentioned antimicrobial peptide stock solution was taken and placed in a 96-well cell culture plate. Then, an equal volume of the test bacterial solution (approximately 10⁵ cells/mL) was added to each well. A positive control (containing the bacterial solution but no antimicrobial peptide) and a negative control (containing neither bacterial solution nor the peptide) were included, respectively. The test bacterial species include Pseudomonas aeruginosa (Pseudomonas aeruginosa PL1, Pseudomonas aeruginosa SP1), Staphylococcus aureus (Staphylococcus aureus 544, Staphylococcus aureus 103), Escherichia coli (Escherichia coli 729, Escherichia coli 112), Klebsiella Pneumoniae (Klebsiella Pneumoniae 106, Klebsiella Pneumoniae 727), Streptococcus pneumonia 383, Acinetobacter baumannii (Acinetobacter baumannii 946, Acinetobacter baumannii 780) (FIG. 1), or Pseudomonas aeruginosa (Pseudomonas aeruginosa PAO1), Staphylococcus aureus (Staphylococcus aureus SP1), and a mixed solution of the above two bacteria (FIG. 2).
C. The culture plate was placed in a constant temperature incubator at 37° C. for 20 h. The minimum inhibitory concentration is determined as the condition where no turbidity is observed at the bottom of the well with naked eyes.

The results in FIG. 1 show that all the antimicrobial peptides of the present invention have a certain broad-spectrum antimicrobial activity, wherein polypeptides Z1 and Z4 have good broad-spectrum antimicrobial activity. The results in FIG. 2 show that polypeptide Z4 has the lowest minimum inhibitory concentration (MIC) against the mixed bacterial solution of Pseudomonas aeruginosa and Staphylococcus aureus (4 μM, which is much lower than that of Indolicidin (ILPWKWPWWPWRR, SEQ ID NO: 5), a well-studied antimicrobial peptide).

### Example 3

### Determination of the Self-Assembly Activity of Polypeptides Z1-Z4:

A. Characterization of the secondary structure of antimicrobial peptides by circular dichroism spectroscopy: The absorption spectra of polypeptides Z1-Z4 were detected at 25° C. using a quartz colorimetric tube (having a diameter length of 1.0 mm) on a Chirascan-plus spectrometer (Applied photophysical, London, England). The spectrum in the range of 195-250 nm were recorded and scanned three times at a scanning speed of 10 nm/min. The mean residue molar ellipticity was calculated from the obtained CD spectra according to the formula ΞM=(Ξobs×1000)/(c×1×n). θM represents the residue molar ellipticity ((deg cm2) dmol-1), θobs is the buffer-corrected ellipticity measured in practice (mdeg), c is the concentration of the antimicrobial peptide (mM), 1 is the optical path length (mm), and n is the number of amino acids.

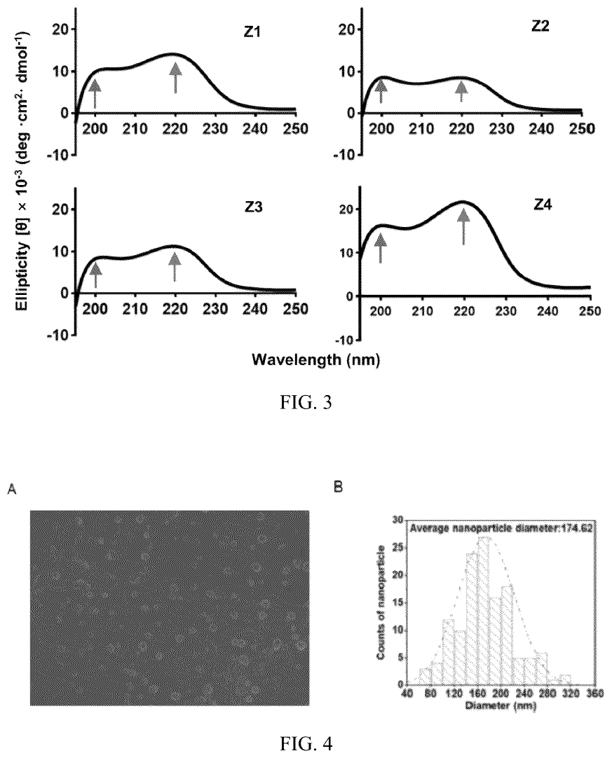

As shown in FIG. 3, all peptides have two positive absorption peaks, located at 200 nm and 220 nm respectively (indicated by arrows), which suggests that the above-mentioned polypeptides may form a supramolecular structure with self-assembly activity.

Without being bound by any theory, it is deduced that the reason why WW++W++WW has better self-assembly activity and MIC than +++WWW+++ is based on the WW at both ends of the peptide, rather than the number of positively charged amino acids (K and/or R).
B. Observation of polypeptide Z4 by scanning electron microscopy (SEM): To prepare SEM samples for observing the self-assembly of the polypeptide, 10 μL of the polypeptide was placed on tin foil that had been ultrasonically treated for 30 min.

After drying, the samples were sputter-coated with gold. All samples were observed using Gimini SEM 500.

As shown in FIG. 4, polypeptide Z4 can self-assemble into a nanomicelle structure in phosphate-buffered saline (PBS) (pH=7.2), with an average particle size of 174.62 nanometers.

### Example 4

### Determination of Bactericidal Activity and Anti-Biofilm Activity of Polypeptide Z4:

A. To determine the bactericidal ability of polypeptide Z4, Pseudomonas aeruginosa and Staphylococcus aureus were suspended in PBS until OD600 nm=0.4, then diluted 1000 times with fresh PBS and mixed in equal volumes. Polypeptide Z4 was added to the bacterial suspension at concentrations of 1×MIC and 2×MIC. The bacterial solution was diluted 10 times, 100 times, and 1000 times at 0, 5, 15, 30, 60, 120, and 180 min, respectively, and 10 μl of each was spotted on LB culture medium. After incubation at 37° C. for 18 h, the single colonies of the microorganisms were counted. The experiment was repeated 3 times and the average value was calculated.

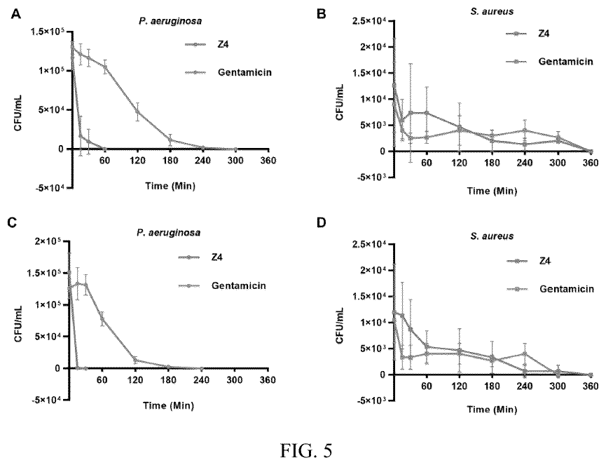

The results showed that at 1×MIC (4 μM), polypeptide Z4 killed all P. aeruginosa and S. aureus within 60 minutes and 360 minutes, respectively (FIG. 5 A-B and Tables 2-3). At 2×MIC (8 μM), polypeptide Z4 killed all P. aeruginosa and S. aureus within 30 minutes and 360 minutes, respectively (FIG. 5 C-D and Tables 4-5). The above results indicate that compared with gentamicin, polypeptide Z4 had a faster bactericidal rate against P. aeruginosa, while there was no significant difference in the bactericidal rate against S. aureus.

TABLE 2

| | Z4 | | | Gentamicin | | | |
|---|---|---|---|---|---|---|---|
| Time | repeat | repeat | repeat | repeat | repeat | repeat | repeat |
| (Min) | #1 | #2 | #3 | #1 | #2 | #3 | #4 |
| 0 | 134000 | 112000 | 104000 | 140000 | 126000 | 130000 | 122000 |
| 15 | 0 | 4000 | 46000 | 116000 | 120000 | 140000 | 108000 |
| 30 | 0 | 0 | 28000 | 104000 | 126000 | 110000 | 126000 |
| 60 | 0 | 0 | 0 | 112000 | 92000 | 106000 | 110000 |
| 120 | 0 | 0 | 0 | 30000 | 52000 | 54000 | 54000 |
| 180 | 0 | 0 | 0 | 12000 | 2000 | 20000 | 12000 |
| 240 | 0 | 0 | 0 | 2000 | 0 | 2000 | 2000 |
| 300 | 0 | 0 | 0 | 0 | 0 | 0 | 0 |

TABLE 3

| | Z4 | | | Gentamicin | | | |
|---|---|---|---|---|---|---|---|
| | repeat | repeat | repeat | repeat | repeat | repeat | repeat |
| Time (Min) | #1 | #2 | #3 | #1 | #2 | #3 | #4 |
| 0 | 4000 | 12000 | 22000 | 4000 | 10000 | 20000 | 2000 |
| 15 | 2000 | 6000 | 10000 | 6000 | 4000 | 4000 | 2000 |
| 30 | 0 | 4000 | 18000 | 2000 | 4000 | 2000 | 2000 |
| 60 | 2000 | 8000 | 12000 | 4000 | 2000 | 0 | 2000 |
| 120 | 2000 | 2000 | 10000 | 2000 | 2000 | 4000 | 8000 |
| 180 | 0 | 2000 | 4000 | 2000 | 4000 | 4000 | 2000 |
| 240 | 0 | 2000 | 2000 | 6000 | 0 | 2000 | 4000 |
| 300 | 2000 | 2000 | 2000 | 4000 | 0 | 2000 | 2000 |
| 360 | 0 | 0 | 0 | 0 | 0 | 0 | 0 |

TABLE 4

| | Z4 | | | Gentamicin | | | |
|---|---|---|---|---|---|---|---|
| | repeat | repeat | repeat | repeat | repeat | repeat | repeat |
| Time (Min) | #1 | #2 | #3 | #1 | #2 | #3 | #4 |
| 0 | 186000 | 136000 | 132000 | 142000 | 134000 | 122000 | 106000 |
| 15 | 2000 | 0 | 0 | 166000 | 104000 | 136000 | 128000 |
| 30 | 0 | 0 | 0 | 116000 | 154000 | 126000 | 130000 |
| 60 | 0 | 0 | 0 | 92000 | 82000 | 70000 | 68000 |
| 120 | 0 | 0 | 0 | 10000 | 22000 | 10000 | 10000 |
| 180 | 0 | 0 | 0 | 4000 | 2000 | 2000 | 0 |
| 240 | 0 | 0 | 0 | 0 | 0 | 0 | 0 |

TABLE 5

| | Z4 | | | Gentamicin | | | |
|---|---|---|---|---|---|---|---|
| | repeat | repeat | repeat | repeat | repeat | repeat | repeat |
| Time (Min) | #1 | #2 | #3 | #1 | #2 | #3 | #4 |
| 0 | 10000 | 22000 | 4000 | 8000 | 12000 | 6000 | 16000 |
| 15 | 16000 | 14000 | 4000 | 0 | 2000 | 2000 | 6000 |
| 30 | 12000 | 12000 | 2000 | 2000 | 0 | 2000 | 6000 |
| 60 | 6000 | 8000 | 2000 | 0 | 6000 | 4000 | 2000 |
| 120 | 8000 | 6000 | 0 | 2000 | 6000 | 0 | 4000 |
| 180 | 6000 | 4000 | 0 | 2000 | 4000 | 0 | 2000 |
| 240 | 2000 | 0 | 0 | 4000 | 6000 | 2000 | 0 |
| 300 | 2000 | 0 | 0 | 0 | 0 | 0 | 0 |
| 360 | 0 | 0 | 0 | 0 | 0 | 0 | 0 |

B. To investigate the anti-biofilm activity of polypeptide Z4, P. aeruginosa and S. aureus were grown overnight at 37° C. in tryptic soy broth medium until the logarithmic phase, and then diluted to 1×107 CFU/mL and 1×106 CFU/mL respectively, and mixed in equal amounts to prepare a mixed bacterial culture. Subsequently, 100 μL of the mixed bacterial suspension was incubated at 37° C. for 24 h to obtain a mixed biofilm. Then, antimicrobial peptides with a final concentration of 2-32 μM were added to a 96-well plate. After incubation at 37° C. for 3 h, the medium was discarded, and the 96-well plate was washed three times with PBS (pH=7.2). After drying at room temperature, 200 μL/well of 0.100 crystal violet staining solution was added to the plate. After incubation at 37° C. for 30 minutes, it was rinsed three times with PBS. After air drying at room temperature, 200 μL/well of 95% ethanol was added and incubated at 37° C. for 30 min, and the absorbance was measured at 595 nm. A total of 3 experiments were set up, and each experiment was repeated 3 times.

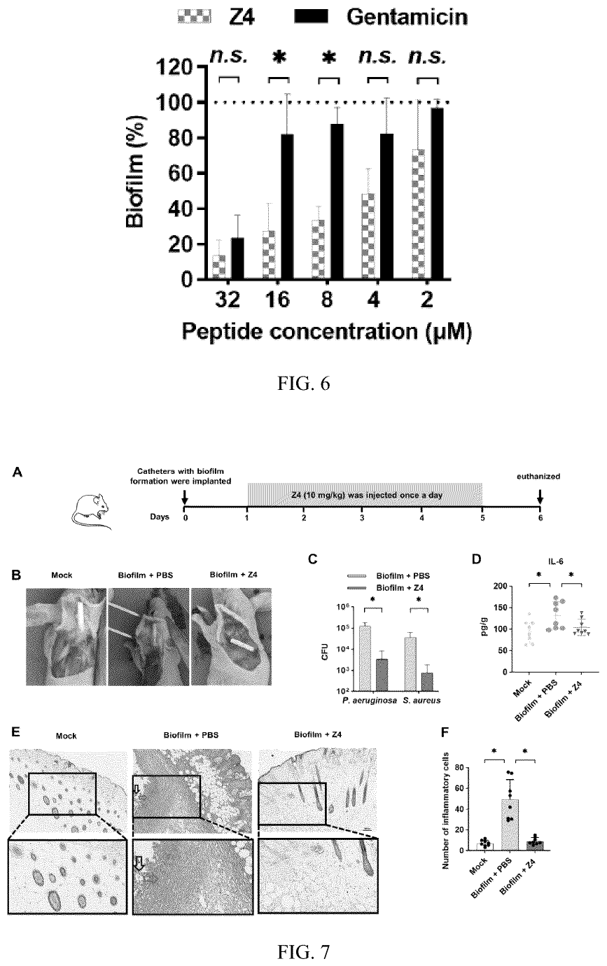

The results are shown in FIG. 6 and Table 6. Polypeptide Z4 can remove more than 50% of biofilm when the concentration reaches 8 μm, which is much better than the 32 M required for gentamicin. This means that polypeptide Z4 has good and direct anti-biofilm activity.

TABLE 6

| | Z4 | | | Gentamicin | | |
|---|---|---|---|---|---|---|
| concentration(μM) | repeat #1 | repeat #2 | repeat #3 | repeat #1 | repeat #2 | repeat #3 |
| 32 | 11.73875 | 6.32539 | 23.0185 | 16.78245 | 38.19119 | 14.79902 |
| 16 | 20.32951 | 16.47543 | 45.18839 | 64.52834 | 107.33 | 74.21549 |
| 8 | 28.15534 | 29.50868 | 42.64257 | 86.97839 | 97.37181 | 78.84672 |
| 4 | 32.15652 | 56.39002 | 56.87797 | 67.84334 | 74.15099 | 105.2663 |
| 2 | 41.62989 | 82.87732 | 95.51086 | 90.78 | 99.17623 | 100 |

### Example 5

### Determination of the In Vivo Antimicrobial Activity of Polypeptide Z4:

The results are shown in FIG. 7. The skin tissue recovery in the polypeptide Z4-treated group was similar to that of the uninfected control catheter (MOCK), while the mice in the control group (treated with PBS only) showed a large amount of pus and tissue damage, consistent with an ongoing infection (FIG. 7B). Moreover, treatment with Z4 significantly reduced the number of Pseudomonas aeruginosa and Staphylococcus aureus colonized in the catheter (FIG. 7C and Table 7). Treatment with Z4 reduced the level of IL-6 in the skin tissue (FIG. 7D and Table 8) and the number of infiltrated inflammatory cells (Table 9, and black arrows in FIGS. 7E and 7F). In summary, the data indicate that polypeptide Z4 has the effects of killing bacteria within the biofilms and reducing the inflammatory response in mice.

TABLE 7

| | Biofilm + PBS | | | | Biofilm + Z4 | | |
|---|---|---|---|---|---|---|---|
| | repeat | repeat | repeat | repeat | repeat | repeat | repeat |
| | #1 | #2 | #3 | #4 | #1 | #2 | #3 |
| P. aeruginosa | 70000 | 80000 | 180000 | 170000 | 900 | 9000 | 200 |
| S. aureus | 10000 | 20000 | 70000 | 50000 | 100 | 2000 | 200 |

TABLE 8

| | Mock | Biofilm + PBS | Biofilm + Z4 |
|---|---|---|---|
| repeat #1 | 65.36667 | 98.3293 | 91.09246 |
| repeat #2 | 136.1654 | 102.6361 | 95.93577 |
| repeat #3 | 64.19128 | 112.8879 | 137.9124 |
| repeat #4 | 79.0256 | 158.6765 | 127.2606 |
| repeat #5 | 104.1886 | 164.7271 | 88.31453 |
| repeat #6 | 113.9905 | 172.8265 | 94.64065 |
| repeat #7 | 114.5841 | 102.0634 | 110.6228 |
| repeat #8 | 87.48666 | 145.2549 | 89.72892 |

TABLE 9

| | Mock | Biofilm + PBS | Biofilm + Z4 |
|---|---|---|---|
| repeat #1 | 4.666667 | 30.33333 | 6.666667 |
| repeat #2 | 6.333333 | 74.66667 | 8 |
| repeat #3 | 7.333333 | 29 | 7.333333 |
| repeat #4 | 5.333333 | 75.66667 | 7.666667 |
| repeat #5 | 11.66667 | 57.33333 | 5.333333 |
| repeat #6 | 9 | 53 | 13 |
| repeat #7 | 10 | 42 | 10.66667 |
| repeat #8 | 3 | 31 | 15 |

### Example 6

### Determination of the Antimicrobial Activity and Anti-Protease Hydrolysis Ability of the Dextrorotatory Isomer of Polypeptide Z4 (Z4-D):

A. The polypeptide Z4 composed entirely of dextrorotatory amino acids is called the dextrorotatory isomer of polypeptide Z4. The polypeptide Z4-D was prepared into a certain stock solution for use. 0.01% acetic acid (containing 0.2% BSA) was used as the diluent, and a series of gradient antimicrobial peptide solutions were prepared in sequence by a two-fold dilution method.
B. 100 μL of the above-mentioned antimicrobial peptide stock solution was taken and placed in a 96-well cell culture plate. Then, an equal volume of the test bacterial solution (approximately 10¹ cells/mL) was added to each well. A positive control (containing the bacterial solution but no antimicrobial peptide) and a negative control (containing neither the bacterial solution nor the peptide) were included, respectively. The test bacterial species include Pseudomonas aeruginosa (Pseudomonas aeruginosa PL1, Pseudomonas aeruginosa SP1), Staphylococcus aureus (Staphylococcus aureus 544, Staphylococcus aureus 103), Escherichia coli (Escherichia coli 729, Escherichia coli 112), Klebsiella Pneumoniae (Klebsiella Pneumoniae 106, Klebsiella Pneumoniae 727), Streptococcus pneumonia 383, and Acinetobacter baumannii (Acinetobacter baumannii 946, Acinetobacter baumannii 780).
C. The culture plate was placed in a constant temperature incubator at 37° C. for 20 h. The minimum inhibitory concentration is determined as the condition where no turbidity is observed at the bottom of the well with naked eyes.

The results in Table 10 indicate that Z4-D has good broad-spectrum antimicrobial activity, similar to the polypeptide Z4 composed entirely of L-amino acids.

TABLE 10

| Strain | MIC (μM) of Z4-D |
|---|---|
| P. aeruginosa PL1 | 16 |
| P. aeruginosa SP1 | 4 |
| S. aureus 544 | 4 |
| S. aureus 103 | 4 |
| E. coli 729 | 4 |
| E. coli 112 | 4 |
| K. pneumoniae 106 | 128 |
| K. pneumoniae 727 | 32 |
| S. pneumoniae 383 | 4 |
| A. baumannii 946 | 4 |
| A. baumannii 780 | 16 |

### Example 7

### Determination of the Anti-Protease Hydrolysis Ability of the Dextrorotatory Isomer of Polypeptide Z4 (Z4-D):

After ultrasonic treatment of polypeptide Z4 and polypeptide Z4-D for 30 min, they were placed at room temperature for 2 h to promote their self-assembly. Subsequently, proteinase K with a final concentration of 0.2 g/mL was mixed with the polypeptide solution (final concentration of 640 μM) and incubated at 37° C. for 0, 30, 60, 90 or 120 min respectively. Then, according to the scheme described in Example 2, the MICs of the polypeptides against the mixed bacterial solution of P. aeruginosa PAO1 and S. aureus SP1 under different treatment conditions were determined.

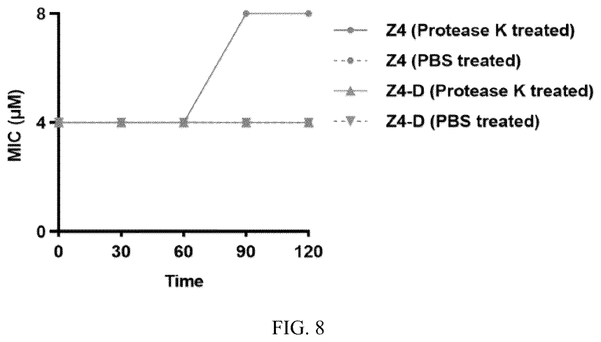

The results are shown in FIG. 8. Within 0-120 minutes of proteinase K treatment, the MIC of polypeptide Z4-D remained unchanged throughout, all at 4 M; while the MIC of polypeptide Z4 increased from 4 μM to 8 μM at 90 minutes, indicating that the antimicrobial activity of polypeptide Z4 decreased by 50% due to hydrolysis by proteinase K, while polypeptide Z4-D showed the ability to resist hydrolysis by proteinase K. This indicates that polypeptide Z4-D will have a longer half-life in vivo than polypeptide Z4, thus having long-acting antimicrobial activity.

The above description of specific embodiments is provided only to help understand the core idea of the present invention. It should be noted that for those of ordinary skill in the art, a number of improvements and modifications may be made to the technical solutions of the present invention without departing from the principle of the present invention, and these improvements and modifications also fall within the scope of protection defined by the claims of the present invention.

### REFERENCES

[1] C. Antimicrobial Resistance, Global burden of bacterial antimicrobial resistance in 2019: a systematic analysis, Lancet 399(10325) (2022) 629-655.
[2] P. S. Stewart, J. W. Costerton, Antibiotic resistance of bacteria in biofilms, Lancet 358(9276) (2001) 135-8.
[3] S. Dehbashi, M. Y. Alikhani, H. Tahmasebi, M. R. Arabestani, The inhibitory effects of Staphylococcus aureus on the antibiotic susceptibility and virulence factors of Pseudomonas aeruginosa: A549 cell line model, AMB Express 11(1) (2021) 50.
[4] G. Orazi, G. A. O'Toole, Pseudomonas aeruginosa Alters Staphylococcus aureus Sensitivity to Vancomycin in a Biofilm Model of Cystic Fibrosis Infection, mBio 8(4) (2017).
[5] D. B. Y. Yung, K. J. Sircombe, D. Pletzer, Friends or enemies? The complicated relationship between Pseudomonas aeruginosa and Staphylococcus aureus, Mol Microbiol 116(1) (2021) 1-15.
[6] A. J. Fischer, S. B. Singh, M. M. LaMarche, L. J. Maakestad, Z. E. Kienenberger, T. A. Pena, D. A. Stoltz, D. H. Limoli, Sustained Coinfections with Staphylococcus aureus and Pseudomonas aeruginosa in Cystic Fibrosis, Am J Respir Crit Care Med 203(3) (2021) 328-338.
[7] C. B. Ibberson, A. Stacy, D. Fleming, J. L. Dees, K. Rumbaugh, M. S. Gilmore, M. Whiteley, Co-infecting microorganisms dramatically alter pathogen gene essentiality during polymicrobial infection, Nat Microbiol 2 (2017) 17079.
[8] N. Dong, Q. Ma, A. Shan, Y. Lv, W. Hu, Y. Gu, Y. Li, Strand length-dependent antimicrobial activity and membrane-active mechanism of arginine- and valine-rich beta-hairpin-like antimicrobial peptides, Antimicrob Agents Chemother 56(6) (2012) 2994-3003.
[9] G. Wang, X. Li, Z. Wang, APD3: the antimicrobial peptide database as a tool for research and education, Nucleic Acids Res 44(D1) (2016) D1087-93.

1. An antimicrobial peptide, wherein the antimicrobial peptide is a short-chain antimicrobial peptide rich in tryptophan and having self-assembly activity.

2. The antimicrobial peptide according to claim 1,
wherein the antimicrobial peptide is a centrally symmetric 9 amino acid peptide, preferably composed of tryptophan (W) and positively charged amino acids, wherein a number of positively charged amino acids is 3-6.

3. The antimicrobial peptide according to claim 2, wherein the positively charged amino acid is selected from arginine (R) and lysine (K).

4. The antimicrobial peptide according to claim 1,
wherein the antimicrobial peptide has a primary symmetric structure with triple tryptophan (WWW) at the center or a tryptophan at the center and double tryptophan (WW) at both ends.

5. The antimicrobial peptide according to claim 1,
wherein the antimicrobial peptide is a broad-spectrum antimicrobial peptide.

6. The antimicrobial peptide according to claim 1, which contains a single tryptophan (W) motif, a double tryptophan motif (WW), a triple tryptophan motif (WWW), or a combination thereof, which is evenly separated by positively charged amino acids (+), preferably the antimicrobial peptide contains the double tryptophan motifs (WW) located at both ends.

7. The antimicrobial peptide according to claim 6,
wherein the antimicrobial peptide is selected from: 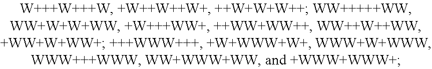
wherein “W” represents tryptophan, and “+” represents a positively charged amino acid, such as arginine (R) or lysine (K), preferably; wherein “+” in the same sequence is the same positively charged amino acid.

8. The antimicrobial peptide according to claim 1, wherein the antimicrobial peptide is selected from any one of SEQ ID NOs: 1 to 4 and a variant thereof containing D-amino acid, preferably SEQ ID NO: 4 (Z4) or Z4-D.

9. A pharmaceutical composition comprising the antimicrobial peptide according to claim 1.

10. Use of the antimicrobial peptide according to claim 1 in preparation of a medicament against microbial co-infections.

11. The use according to claim 10, wherein the microbial co-infections are co-infections of Pseudomonas aeruginosa-Staphylococcus aureus.

12. The antimicrobial peptide according to claim 2, wherein the amino acid peptide comprises L-amino acids and/or D-amino acids.

13. The antimicrobial peptide according to claim 2, wherein the number of positively charged amino acids is 4.

14. The antimicrobial peptide according to claim 2, wherein the number of positively charged amino acids is 6.


SEQUENCE LISTING (5 sequences)

SEQ ID NO: 1 - protein, 9 aa, synthetic construct
KWKWWWKWK

SEQ ID NO: 2 - protein, 9 aa, synthetic construct
RRRWWWRRR

SEQ ID NO: 3 - protein, 9 aa, synthetic construct
WWRRWRRWW

SEQ ID NO: 4 - protein, 9 aa, synthetic construct
WWKKWKKWW

SEQ ID NO: 5 - protein, 13 aa, synthetic construct
ILPWKWPWWPWRR